(19) United States
(12) Patent Application Publication
(10) Pub. No.: US 2023/0211119 A1
(43) Pub. Date: Jul. 6, 2023

(54) A Urinary Catheter

(71) Applicant: Coloplast A/S, Humlebaek (DK)

(72) Inventor: Troels Gottfried Pedersen, Gilleleje (DK)

(21) Appl. No.: 17/927,701

(22) PCT Filed: May 26, 2021
(86) PCT No.: PCT/DK2021/050163

§ 371 Date: Nov. 24, 2022

(30) Foreign Application Priority Data
May 27, 2020 (DK) PA 2020 70342

(51) Int. Cl. A61M 25/00; A61M 25/04

(52) U.S. Cl. CPC A61M 25/0017; A61M 25/04; A61M 25/007

(57) ABSTRACT

Disclosed is a urinary catheter (1) for indwelling use. The catheter is provided with multiple small drainage openings (6) at a drainage portion (7). This will reduce blockage of the drainage openings during the dwelling period. The catheter is provided with either more than 11 drainage openings or with drainage openings each having a cross- sectional area of less than 2 mm2

The invention relates to a urinary catheter and in particular to a urinary catheter for indwelling use.

### SUMMARY OF THE INVENTION

The disclosure concerns a urinary catheter for indwelling use having multiple small drainage openings. Such a catheter will have a reduced tendency to blockage during the dwelling period.

### BRIEF DESCRIPTION OF THE DRAWING

The accompanying drawings are included to provide a further understanding of embodiments and are incorporated into and are part of this specification. The drawings illustrate embodiments and together with the description serve to explain principles of embodiments. Other embodiments and many of the intended advantages of embodiments will be readily appreciated as they become better understood by reference to the following detailed description. The elements of the drawings are not necessarily to scale relative to each other. Like reference numerals designate corresponding similar parts.

FIG. 1 illustrates a cross-sectional view of a catheter having multiple small drainage openings. The catheter is illustrated as positioned in the bladder.

FIG. 2 illustrates a perspective view of a catheter positioned in the bladder.

FIG. 3 illustrates a catheter with an expansive retaining element and drainage openings positioned in the expansive element.

FIG. 4 illustrates a catheter with an expansive retaining element, the drainage openings are positioned in proximal drainage portion.

FIG. 5 illustrates a catheter with a bend for retaining the catheter in the bladder, the drainage openings are positioned in a proximal drainage portion.

FIG. 6 illustrates a catheter with three arms for retaining the catheter in the bladder, the drainage openings are positioned in the three arms.

FIG. 7 illustrates a catheter with a spiral-shaped retaining element, the drainage openings are positioned in a drainage portion in the spiral-shaped retaining element.

FIGS. 8A and 8B illustrate a valve, which can be used to close off the distal end of an interior lumen of the catheter.

FIG. 9 shows curves over how the flow converges.

FIGS. 10 and 11 illustrate a prior art catheter and the problems associated with that.

### DETAILED DESCRIPTION

Embodiments relate to a urinary catheter having a tip portion with a tip in a proximal insertion end of the catheter, a tubular portion extending from the tip portion to a distal outlet end; the tubular portion having an interior lumen, the catheter being provided with a drainage portion provided with drainage openings configured for allowing urine to drain into the interior lumen of the tubular portion, the drainage portion being provided with more than 11 drainage openings; the catheter being configured for sitting in the urethra with at least the tip portion and the drainage portion in the bladder for a prolonged period of time.

Embodiments relate to a urinary catheter having a tip portion with a tip in a proximal insertion end of the catheter, a tubular portion extending from the tip portion to a distal outlet end; the tubular portion having an interior lumen, the catheter being provided with a drainage portion provided with drainage openings configured for allowing urine to drain into the interior lumen of the tubular portion, the drainage portion being provided with drainage openings each having a cross-sectional area of less than 1 mm²; the catheter being configured for sitting in the urethra with at least the tip portion and the drainage portion in the bladder for a prolonged period of time.

A catheter as described above will provide a very minimal local suction on the bladder tissue due to the sheer number of drainage openings and the small size of the drainage openings.

A catheter as defined above sits in the urethra with at least the tip portion in the bladder for a prolonged period of time. In some situations, the catheter may sit for a period of up to 30 days or longer; this is usually and will also in the following be referred to as indwelling use of the catheter with continuous emptying. In other situations, the catheter may sit for a shorter period of time, such as 1 day or 2 days. This will in the following be referred to as semi-dwelling use with continuous emptying. Furthermore, the catheter may sit for a prolonged period of time but will be emptied intermittently. This will in the following be referred to as a catheter with intermittent emptying.

When a catheter sits indwelling in a bladder and is used for continuous flow, there is a constant dripple or limited flow of urine from the bladder into the catheter through the eyelets or drainage openings, through the interior lumen of the catheter and out through the distal outlet end. In most cases the distal outlet end will be connected to a urine collecting bag through a tube or hose both of which are well-known in the art. Because the urine collecting bag in most situations will be positioned lower than the bladder — maybe 10-30 or even 50 cm lower — this height difference will provide a constant sub-pressure at the eyelets or drainage openings, thus allowing any urine in the bladder to flow through the catheter in the correct direction, i.e. outwards from the bladder.

This sub-pressure will provide a suction effect of the eyelets or drainage openings in the bladder. During use of a prior art indwelling catheter having two large eyelets, a situation may occur where one of the eyelets get into contact with the bladder wall. The tissue on the bladder wall is very soft and compliant and may in some situations be sucked partly into the eyelet, if the eyelet is large enough. This will lead to a blockage of the eyelet, in which case the urine may not be able to be drained properly from the bladder. If both eyelets should be blocked, the catheter would need to be repositioned before drainage of urine could continue. This is due to the suction effect provided by the catheter, which makes it highly unlikely that the bladder wall tissue will be released from the eyelets without repositioning of the catheter. Instead a situation of steady suction will be maintained until the catheter is repositioned by outside forces. Furthermore, the suction effect of those rather large eyelets openings may in this situation influence the bladder wall and may over time lead to trauma to the bladder wall.

Providing drainage openings of a small size as compared to the eyelets of prior art catheters will alleviate or at least minimise this problem. This is due to the fact that the factor between the cross-sectional area and the circumference of the drainage opening influences this. Minimising this factor will reduce the influence of the negative pressure (suction) at the bladder tissue — see below.

Furthermore, if multiple drainage openings are provided (12 or more), it will be close to impossible to have a blockage of all of the drainage openings at the same time, therefore at least some drainage openings will be able to provide draining of urine. These multiple drainage openings will also alleviate the need for repositioning of the catheter, because there will always be at least one non-blocked opening, which will minimise the risk of a situation of steady suction occurring.

The same problems and advantages as mentioned above, will also apply to a catheter used for semi-dwelling use with continuous emptying, i.e. when the catheter sits in the urethra with the tip portion in the bladder for a prolonged period of time of 1-2 days. Even though the use in these situations are shorter than for indwelling use, there is still a risk that if only 2 drainage openings (or eyelets) are used, as in prior art catheters, then these eyelets may become blocked during the continuous flow and provide a situation of steady suction.

A well-known phenomenon with prolonged use of indwelling or semi-dwelling urinary catheters is encrustation. This is typically due to P. Mirabilis bacteria being positioned at the outer and inner surfaces of the catheter. These bacteria may be introduced in the bladder with the catheter or may travel along the catheter up through the urethra during the dwelling period. In some cases, these encrustations may partly block the drainage openings and may limit the flow through the catheter.

The influence of encrustation may be minimised due to the catheter having multiple drainage openings. This is because it is less likely that encrustation over multiple drainage openings will occur. The multiple drainage openings of the catheters disclosed herein will cover a rather large surface area of the catheter.

When using prior art indwelling catheters, situations may occur, where the tip pokes and irritates the mucosa of the bladder, because the catheter (and thus the tip) may move around in the bladder during the dwelling period. A catheter as disclosed herein will have a very soft and flexible tip portion and therefore the tip will be less likely to irritate the mucosa of the bladder during the dwelling period.

In the following, whenever referring to a proximal end of an element of the invention, the referral is to the end adapted for insertion. Whenever referring to the distal end of an element, the referral is to the end opposite the insertion end. In other words, the proximal end is the end closest to the user, when the catheter is to be inserted and the distal end is the opposite end -the end furthest away from the user when the catheter is to be inserted.

The longitudinal direction is the direction from the distal to the proximal end. The transverse direction is the direction perpendicular to the longitudinal direction, which corresponds to the direction across the catheter.

The urinary catheter according to the disclosure comprises a main tubular portion extending from a tip portion in the proximal insertion end to a distal outlet end. The tubular portion can be cylindrical or conical. In embodiments, the tubular portion has an oval cross-section. The tubular portion is configured for providing urine flow in an interior lumen through the catheter. A tip portion with a tip at the proximal end is positioned in the proximal portion of the catheter. The catheter is provided with a drainage portion, which includes multiple drainage openings providing for flow of urine between the outside of the catheter and the interior lumen of the tubular portion. In embodiments, the drainage portion is longer than the typical flow zone on a prior art catheter, where the flow zone is defined as the length from the distal edge of the distal eyelet to the proximal edge of the proximal eyelet. In embodiments, the catheter comprises a connector in the distal end. In an embodiment the connector comprises a flared end of the catheter so that the diameter of the connector increases with respect to the tubular portion.

Usually, indwelling urinary catheters are from size 12 FR to size 30 FR. FR (or French size or Charriere (Ch)) is a standard gauge for catheters approximately corresponding to the outer circumference in mm. More accurately, the outer diameter of the catheter in mm corresponds to FR divided by 3. Thus 12 FR corresponds to a catheter with an outer diameter of 4 mm and 30 FR corresponds to a catheter with an outer diameter of 10 mm.

To serve as examples of the embodiments disclosed, the material used for the catheter may be conventional TPU (Thermoplastic polyurethane) or in general TP. Further the material may be more traditional plastic used in indwelling catheters, such as Silicone or PVC. To provide a gentle and soft surface the catheter may further have more layers of plastics, in which the outer layer may be more flexible than the inner. This is also known from traditional indwelling catheters, where the outer layer may be latex or another more flexible and elastic material that allows inflation forming a retention balloon (Foley catheter).

In addition, the material selection for such a catheter may vary into several groups of material including bioplastics and materials refined from naturally removable resources such as plant starks or fibres from vegetables.

In addition, the surface of the catheter may be adapted to minimise discomforting during the insertion period and replacement. Such surface may in addition provide an antibacterial surface, preventing e.g. P. Mirabilis or other bacteria to migrate along the catheter into the bladder. To serve as an example, the surface may have antibacterial properties due to chemical properties, e.g. using silver. In yet another embodiment, the surface may have electric properties that allows the catheter to perform electric potentials between different areas in close proximity, which is a known mean for inhibiting bacterial bio-film growth. As an example, silver particles in combination with other metallic particles may be used as a surface layer having electric properties.

The catheter disclosed may have a lubrication that is used to minimize discomforting during insertion. The lubrication may be in the form of a hydrogel, which is put on the exterior surface of the catheter prior to insertion. Such lubrication may also have properties that make it less likely for bacteria to attach to the surface, thus preventing that bacteria being pushed further up and into the bladder during replacement of the catheter.

Drainage openings in catheters, including the ones described herein, are sometimes in the art referred to as eyelets or eyes. The drainage openings in the catheters in this disclosure goes through a wall in the tubular portion of the catheter from an exterior surface of the tubular portion to the interior lumen, such that urine can travel from outside of the catheter to the interior lumen through the drainage openings. In the exterior surface, the drainage openings have a closed loop circumference and may be circular, oval, square, triangular and any other closed loop shape. In embodiments, the drainage openings are star-shaped.

Embodiments relate to a urinary catheter having a tip portion with a tip in a proximal insertion end of the catheter, a tubular portion extending from the tip portion to a distal outlet end; the tubular portion having an interior lumen and a drainage portion provided with drainage openings configured for allowing urine to enter the interior lumen, the drainage portion being provided with more than 11 drainage openings; the catheter being configured for sitting in the urethra with at least the tip portion in the bladder for a prolonged period of time, wherein the distal end of the interior lumen is provided with a closure configured for closing off the interior lumen, which closure is provided with a valve configured for allowing draining of urine intermittently from the catheter.

Such a catheter may alleviate the need for using a urine collecting bag, because the urine may be emptied directly into a toilet by opening the valve.

Embodiments relate to a method of draining urine from the bladder using an indwelling catheter having a tip portion with a tip in a proximal insertion end of the catheter, a tubular portion extending from the tip portion to a distal outlet end; the tubular portion having an interior lumen and a drainage portion provided with drainage openings configured for allowing urine to enter the interior lumen, the drainage portion being provided with more than 11 drainage openings; the catheter being configured for sitting in the urethra with at least the tip portion in the bladder for a prolonged period of time, wherein the method comprises the step of inserting the catheter through the urethra and leaving the catheter with the tip portion in the bladder for a prolonged period of time.

The catheter may in embodiments be provided with a closure for closing off the interior lumen, the closure further comprising a valve allowing for draining of urine through the valve in an open position, while the draining of urine is not possible when the valve is in a closed position.

The method may comprise the step of keeping the valve closed for a predefined period of time, such as for 3-4 hours or slightly more, opening the valve for draining the urine collected in the bladder during the predefined period of time and subsequently closing the valve when the bladder is empty. These steps may be repeated throughout the dwelling time of the catheter with shorter or longer intervals between the emptying of the bladder by opening the valve.

There are several factors determining the flow and pressure in a urinary catheter. One factor is the resistance to flow in the tubular portion of the catheter and another factor is the resistance to flow in the drainage openings. These two factors together will provide a combined resistance to flow. In the context of this disclosure, the resistance to flow in the tubular portion is known as R1 and the resistance to flow in the drainage openings is known as R2. The combined effect is known as R.

The flow and pressure in a urinary catheter depends on the pressure difference between the bladder and the ambience as well as on the flow through the catheter. The bladder pressure depends on the detrusor pressure and the abdominal pressure. The volumetric flow through the catheter will provide a suction effect on the urine in the bladder because the urine, being mostly water, is incompressible and thus the volume flowing out of the catheter will correspond to the volume flowing into the catheter.

The suction ability is related to the volumetric flow rate through the catheter. The relation between suction ability and the volumetric flow rate is due to the volumetric flow being dependent (amongst others) on the difference in pressure from the inlet of the flow to the outlet of the flow. During draining with a urinary catheter, this pressure difference corresponds to the loss of static pressure between the bladder-pressure and the lumen-pressure at the drainage openings. The resistance to the flow equals the pressure loss in the catheter divided by the volumetric flow rate.

$${R}_{1} = \frac{Δ P}{Q} = \frac{8 μ ⋅ L}{π ⋅ {a}^{4}}$$

In the above equation, R1 is the resistance to flow, ΔP is the pressure loss, Q is the volumetric flow rate, µ is the viscosity in the fluid, L is the length of the flow and a is the area over which, the flow occurs. This means that “a” depends on whether the interior lumen is filled or not. If the interior lumen is filled, then “a” corresponds to the cross-sectional area of the interior lumen of the catheter. If it is not filled, then “a” is the cross-sectional area of the filled part of the interior lumen. The volumetric flow rate of the lumen can then be expressed as follows:

$$Q = \frac{Δ P ⋅ π ⋅ {a}^{4}}{8 μ ⋅ L}$$

The above equation can be used to describe the flow in an interior lumen of a tubular portion of the catheter.

As appears from above, the flow, and thereby the resistance to flow, R1, through the interior lumen will be different depending on whether the bladder is continuously drained or it is allowed to fill and then drain intermittently.

With respect to the resistance to flow into the drainage openings, one of the determining factors, as mentioned above, is the viscous friction between the urine and the catheter wall through which the drainage opening extends. In other words, R2. This means that the surface area of each opening influences the inflow from the bladder to the lumen. The surface area is determined as follows:

$${A}_{s u r f a c e} = C i r c u m f e r e n c e ⋅ t$$

Where t is the thickness of the catheter wall. A prior art catheter having two larger drainage openings (2.5 by 1.1 mm) will have a total inflow area of 5.5 mm². A urinary catheter having 11 drainage openings each with a diameter of 0.8 mm will also have a total inflow area of 5.5 mm². This means that these two catheters should be able to drain the same amount of urine over time. However, the total surface area of the drainage openings is markedly different. For the prior art catheter, the total surface area is 14 mm (the sum of the circumference of two drainage openings of 2.5 by 1.1 mm) times the catheter thickness, whereas the same value for the urinary catheter having 11 smaller drainage openings is approximately 28 mm (the total circumference of 11 drainage openings each having a circumference of 2.5 mm) times the catheter thickness. If the catheter thickness is the same, this illustrates that the total surface area of the intermittent urinary catheter having 11 small drainage openings are twice the total surface area of a prior art catheter. This means that the resistance due to viscous friction is markedly higher in the urinary catheter with small drainage openings — and thereby, all of the drainage openings are “forced” to contribute to a larger extent to give a more even distribution of flow and pressure across the external surface of the catheter as well as in a distance close to the catheter. In other words, the flow resistance caused by viscous friction between urine and the catheter wall, through which the drainage openings extend, is increased as the size of the drainage openings decreases. This is due to the fact that there are more drainage openings — hence a larger area, which the urine is in contact with during voiding. This is in spite of the total drainage area. Changing the distance, size and shape of openings with respect to a position higher or lower on the catheter makes it possible to design how evenly the flow is distributed. Longer and less concentration of openings closer to the bladder neck will increase the flow higher up on the catheter. In similar ways, smaller drainage openings lower down will increase the flow higher up during voiding.

If the bladder is continuously drained, then the flow-rate through the catheter will be low, but continuous. This means that the interior lumen in the catheter will only rarely fill and the inflow rate into the drainage openings will be low. However, even this low inflow rate will be influenced by the resistance to flow across the drainage openings, R2. Therefore, smaller drainage openings will lead to a better distribution of flow across the drainage openings. In a prior art catheter having only large drainage openings with small resistance to the flow across the drainage openings, a situation may occur where only one of the drainage openings (or eyelets) contribute to the draining and the other drainage openings are not used. Therefore, smaller drainage openings may also lead to a better emptying of the bladder, because drainage openings over a larger exterior surface of the catheter contributes to the draining.

If the bladder is drained intermittently, for example in situations where the catheter is provided with a closure at the distal end, then the interior lumen in the catheter will be filled during voiding of the bladder. Furthermore, the resistance to flow across the drainage openings will also influence the total resistance in a similar way as explained above under continuously draining of the bladder.

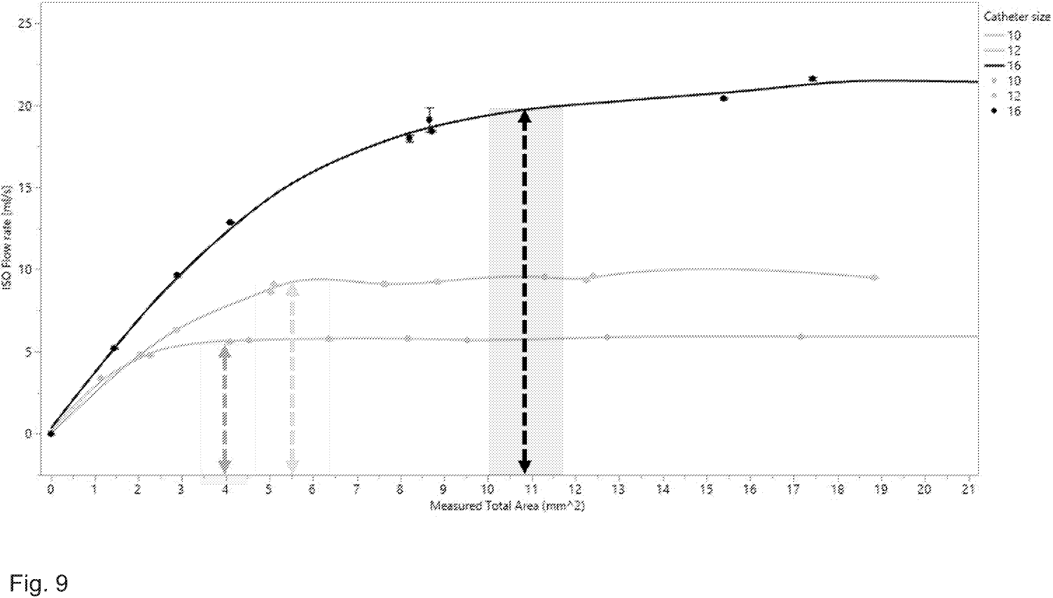

During voiding, the inflow into the urinary catheter will increase with the number of drainage openings of a certain size until a maximum flow-rate is reached. In other words, the inflow will increase with the total cross-sectional area of the drainage openings. The maximum flow-rate is determined by the tubular flow resistance in the interior lumen of the catheter, which at a certain point becomes dominant. This is further illustrated in FIG. 9, where Q, the volumetric flow rate, is shown as a function of the total cross-sectional area of the drainage openings. The figure illustrates that Q increases up to a certain point, depending on the lumen of the catheter and from there, the total cross-sectional area seems to lose influence on the flow. If drainage openings of a certain size, e.g. 0.7 mm in diameter, are used, then there would be a similar dependency between Q and the number of drainage openings. This means that adding further drainage openings will not lead to an increase in the volumetric flow-rate.

Embodiments relate to the tip of the catheter being a closed tip of the tip portion. A typical type of closed tip is a nelaton tip.

Embodiments relate to the tip of the catheter being open. In these situations, the tip may have a side-opening extending to the tip or, alternatively, be completely open, such as a cut-off tube.

Embodiments relate to a urinary catheter as described above, the urinary catheter further having retention means. Embodiments also relate to a method as described above, wherein the method further comprises activating the retention means.

By retention means is meant means for self-retaining the catheter inside the bladder. When retention means are used, any need for fixing the catheter outside the urinary system may be alleviated. If retentions means are not provided, users may have to have the catheter taped or otherwise fixed to an area of the body close to the urethral entrance e.g. to the leg of the user. This fixing may be less comfortable for the user.

A catheter including retention means may comprise a tubular portion extending from the distal end towards the proximal end.

Embodiments relate to the retention means being in the form of an inflatable balloon. In these embodiment, the inflatable balloon and an exterior surface area of the catheter at the position of the balloon may together define a balloon lumen. The balloon lumen may increase by inflating the balloon element through a second interior lumen extending through the catheter from its distal end to an outlet at the outer surface of the catheter covered by the balloon. At the distal end the second inside lumen may terminate in an inlet configured for cooperating with a syringe or like means to fill the balloon lumen — typically with liquid.

Embodiments relate to a method as described above, wherein an activation of the retention means involves inflating a balloon element through a second interior lumen. The method may further involve using a liquid-filled syringe to inflate the balloon element.

Embodiments relate to the retention means being in the form of an expansive part of the catheter positioned just distally of the tip portion. By just distally of the tip portion is meant within the first 5 mm of the tip portion.

The expansive part may be provided by dragging a thin wire inside the lumen that influences a section of the catheter where the material or wall section differs from the normal tube-shaped catheter, so that the tension influences the forming. In one embodiment, the catheter section being deformed shapes into a mushroom like form, providing a retention of the catheter.

Embodiments relate to the drainage openings being positioned in the expansive part of the catheter, meaning that the drainage openings may in some situations be positioned in the retention means.

Embodiments relate to the retention means being in the form of a bendable portion just distally of the tip portion. A bendable portion allows the tip portion to be bend and thus functions as retention means.

The bendable portion may be provided by use of a preformed catheter which due to flexibility of the material and pre-shaping can be inserted into the urethra and bladder without discomfort, but once reaching the bladder, the catheter will bend and perform a retention functionality. In addition, the catheter may have a build-in wire in one side of the catheter wall or lumen, providing a tension in one side of the catheter once inserted, the said wire being tensioned by the user or operator providing a bending of the catheter or part of the catheter.

Using several wires makes it possible to bend partial portions of the proximal catheter end, allowing multiple minor tubes to form a retention means for keeping the catheter in the bladder and ensuring a distributed drainage.

Embodiments relate to the retention means being in the form of a spiral-shaped tip portion. In one embodiment, the spiral shape is provided by pre-forming part of the catheter, given the stiffness of the catheter is so low, that the catheter may be positioned and removed without discomfort for the user/patient. As a variant the catheter may have an internal rod which straightens out the catheter during insertion/removal, but once removed, the catheter curls in a dedicated section, providing sufficient retention of the catheter.

Embodiments relate to the retention means being in the form of a flower-shaped tip portion. The flower-shaped tip portion may have two, three, four or more arms. The arms are provided as bendable portions just distally of the tip portion. By just distally is meant within the first 5 mm in the longitudinal direction of the catheter. This means that the catheter extends as a tubular portion from the distal end towards the tip portion. In the transition between the tubular portion and the tip portion, the catheter is divided into 2, 3, 4 or more proximally extending arms, which constitute the tip portion. The drainage openings are provided in the arms and are preferably distributed between all arms. However, the drainage openings may also be provided in only one arm or distributed over two or three of more arms.

The arms serve two purposes. Firstly, they distribute the draining area over a rather large area of the bladder, and secondly, the arms function as retention means.

The arms may be 20 or 30 mm long or longer such as 35 mm or 40 mm.

Embodiments relate to a catheter configured for sitting in the bladder for a period of 1-2 days -and shorter than 3 days. This means that in these embodiments the prolonged period of time is between 1-3 days.

Embodiments relate to a catheter configured for sitting in the bladder for a period of up to four weeks or more, e.g. for up to 30 days. This means that in these embodiments, the prolonged period of time is up to 30 days.

Embodiments relate to the catheter being provided with more than 20 drainage openings.

Embodiments relate to the catheter being provided with more than 30 drainage openings.

Embodiments relate to the catheter being provided with more than 50 drainage openings.

Embodiments relate to the catheter being provided with more than 100 drainage openings.

Embodiments relate to a catheter having drainage openings having a cross-sectional area of less than 2 mm². Drainage openings may be provided with a circular opening in the exterior surface of the catheter. In this case, the diameter of the drainage openings may be less than 1.6 mm. For other configurations of drainage openings, a largest dimension across the opening may be less than 1.6 mm. This means that, for example, if the drainage opening is a rectangular opening in the exterior surface of the catheter, then the diagonal is less than 1.6 mm.

Embodiments relate to a catheter having multiple drainage openings, wherein each drainage opening has a cross-sectional area of less than 0.8 mm². As examples, this corresponds to circular drainage openings with a diameter of less than 1 mm or for other configurations, drainage openings with a largest dimension (e.g. diagonal) of less than 1 mm.

Embodiments relate to a catheter having drainage openings with a cross-sectional area of 2 mm² and drainage openings with a cross-sectional area of 1 mm². For example, the catheter may have 12 drainage openings, 6 of them with a cross-sectional area of 2 mm² and 6 of them with a cross-sectional area of 1 mm². A combination of larger and smaller drainage openings may be an advantage with respect to distribution of flow over the exterior surface of the catheter.

In an embodiment, the drainage openings are positioned in a scattered manner in the longitudinal direction as well as around the circumference of the urinary catheter.

In an embodiment, the drainage openings are positioned in four longitudinal rows with 90 degrees between them around the circumference of the urinary catheter.

In an embodiment, the drainage openings are positioned in 6 (six) longitudinal rows with 60 degrees between each row around the circumference of the urinary catheter.

In an embodiment, the drainage openings are positioned in 8 (eight) longitudinal rows with 45 degrees between each row around the circumference of the urinary catheter.

In an embodiment, the drainage openings are positioned in two longitudinal rows with 180 degrees between the rows around the circumference of the urinary catheter.

In an embodiment, the drainage openings are positioned in two pairs of parallel rows with 180 degrees between pairs of rows around the circumference of the urinary catheter.

In an embodiment, the drainage openings are dispersed according to a helical distribution around the circumference of the urinary catheter.

In an embodiment, the drainage openings are dispersed in at least two rows, such that the drainage openings in two neighbouring rows are staggered with respect to each other around the circumference of the urinary catheter.

In an embodiment, the drainage openings are positioned randomly distributed at the exterior surface of the catheter.

Having an increased number of directions provide for better inflow and decreased risk of bladder tissue blocking contact with all drainage openings.

In an embodiment, the drainage openings are positioned so that the distance between them is smaller towards the proximal end of the drainage portion than the distance between them at the distal end of the drainage portion. This provides an increased inflow area and thereby increased inflow options towards the proximal end of the catheter. The urine in the bladder will drain through the drainage openings having the smallest pressure difference from the drainage position to the storage position of the urine. This will typically mean that the urine predominantly will drain through the lower-most positioned drainage openings, i.e. the most distal drainage openings — simply due to the reduced height difference as compared to the most proximal drainage openings. Providing a large inflow area towards the proximal end of the catheter leads to a better distribution of the flow between the drainage openings.

The total inflow area may be defined as the sum of the cross-sectional areas of the drainage openings. This total inflow area is preferably higher than the sum of the cross-sectional areas of two standard eyelets.

Embodiments relate to a catheter as described above, with a closure in the form of a valve as described above, wherein the valve is of a type being a ball-valve, seat valve or butterfly valve. Any other type of suitable valve may be used — the above are only mentioned as examples. The valve may be operated by a lever or other physical means from the outside of the valve-housing. Alternatively, the valve may be electric and may be operated by a solenoid.

### DETAILED DESCRIPTION OF THE DRAWING

Embodiments, and features of the various exemplary embodiments described in this application, may be combined with each other (“mixed and matched”), unless specifically noted otherwise.

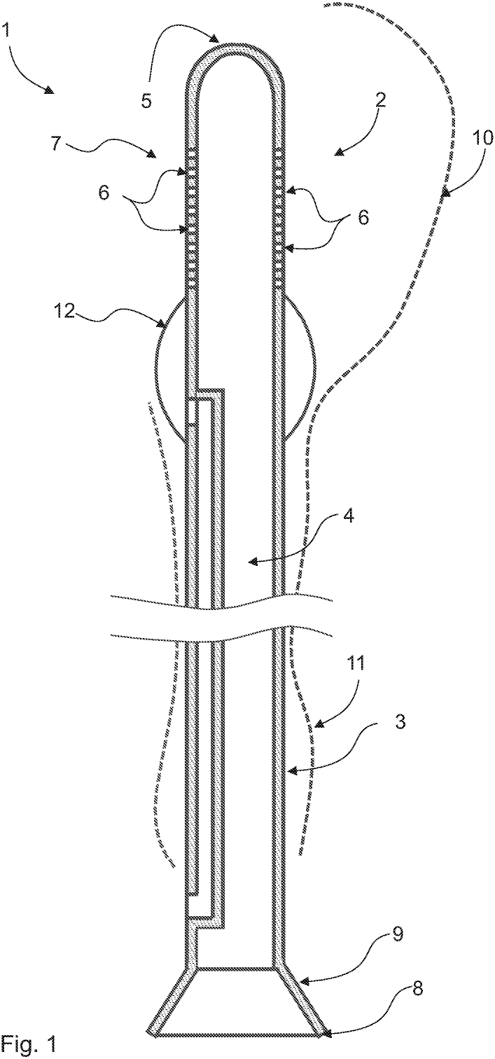

FIG. 1 illustrates a cross-sectional view of an embodiment of a catheter 1 as described herein. The catheter 1 has a tip portion 2 in the proximal portion of the catheter, the tip portion 2 is configured for being positioned in the bladder 10, while the catheter 1 dwells in the user. The catheter further has a tubular portion 3, which in the illustrated embodiment is configured for being positioned in the urethra 11 while the catheter sits in the user. Alternatively, the tubular portion may be configured for extending through the abdominal wall, in case a suprapubic catheter is provided. Irrespectively of whether the use is for positioning in the urethra or through the abdominal wall, the tubular portion 3 is provided with an interior lumen 4 allowing for draining of urine from the bladder 10. The tip portion 2 has a tip 5 in a proximal end of it. In the illustrated embodiment, the tip 5 is a closed tip (a nelaton tip), but may alternatively be an open tip, a bend tip (Tiemann tip), a bulb-shaped tip or any other type of tip known in the art of urinary catheters. The catheter as described herein is provided with multiple drainage openings 6 in a drainage portion 7 of the catheter. In the illustrated embodiment, the drainage portion 7 is constituted as a portion of the tip portion 2. The catheter is further provided with retention means in the form of an inflatable balloon 12. Towards the distal end 8 of the catheter, the catheter is in the illustrated embodiment provided with a connector 9 configured for connecting the catheter to a drainage tube (not shown) and thus eventually to a collecting bag (not shown).

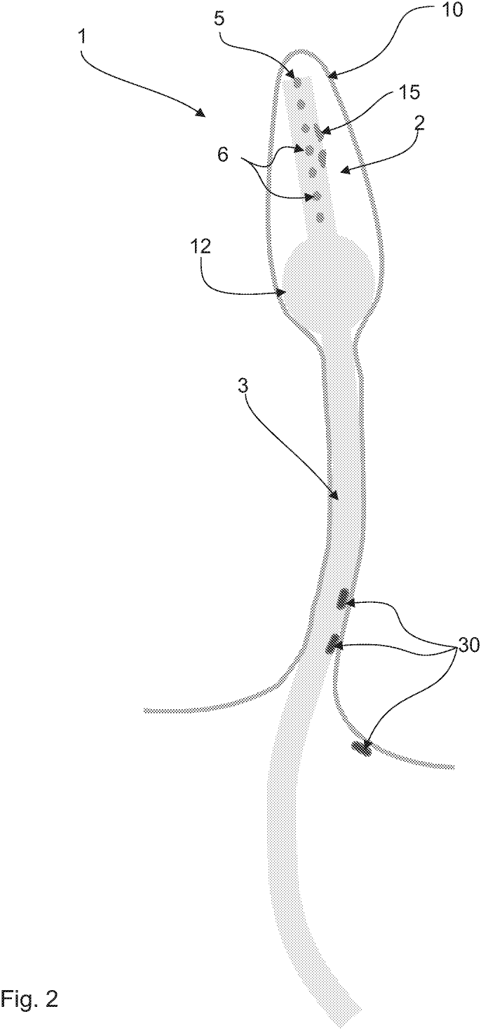

FIG. 2 illustrates a perspective view of an embodiment of a catheter 1 as described herein. The catheter has a tip portion 2 sitting in the bladder 10 while the catheter dwells in the user. In the illustrated embodiment, the tip portion has a tip 5, which is open. Furthermore, as in the embodiment in FIG. 1, the catheter is provided with a drainage portion 7 in the tip portion, and the drainage portion 7 is provided with multiple drainage openings 6. Also, in the embodiment in FIG. 2, the catheter is provided with retention means 12 configured for keeping the catheter in place with the tip portion in the bladder. The retention means 12 is, in the illustrated embodiment, an inflatable balloon which is inflated when the balloon is positioned in the bladder. Encrustations 15 are illustrated on an exterior surface of the catheter. This is a common problem with indwelling urinary catheters. However, because of the multiple drainage openings 6, blocking of all drainage openings is unlikely to happen in catheters as described herein. Bacteria, such as P. Mirabilis, may travel up through the urethra during the dwelling period of the catheter, and this may influence the encrustations on the exterior surface of the catheter. In the illustrated embodiment, this is indicated at 30.

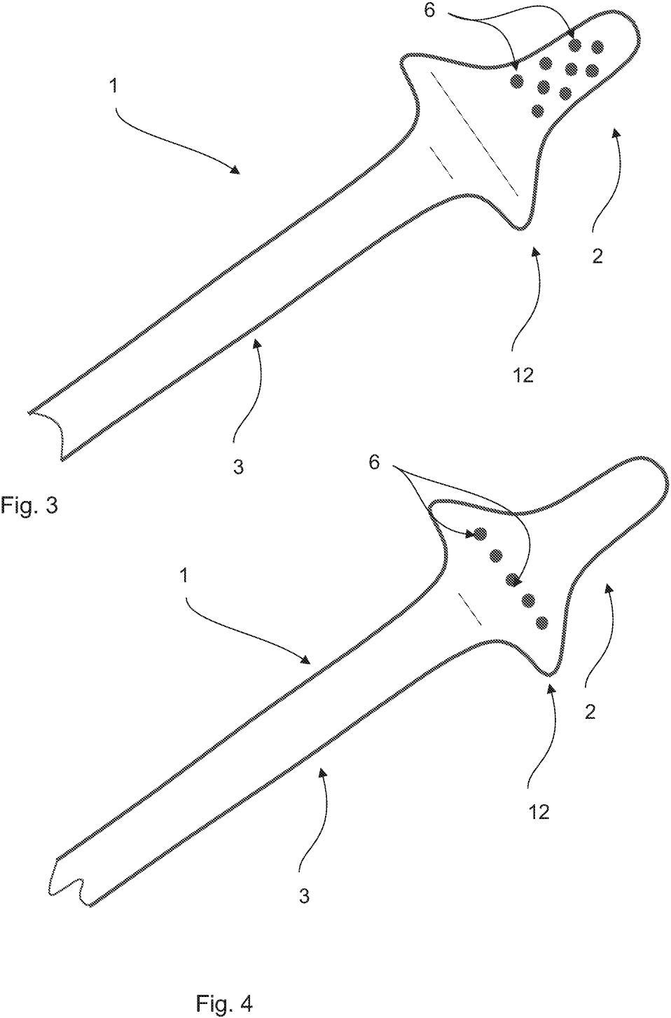

FIGS. 3 and 4 illustrate embodiments of a urinary catheter provided with retention means 12. In the embodiments of FIGS. 3 and 4, the retention means are in the form of an expansive part of the catheter. The urinary catheter is also provided with a tubular portion 3, a tip portion 2 and drainage openings 6. In the embodiment of FIG. 3, the drainage openings 6 are positioned in the tip portion 2. In the embodiment of FIG. 4, the drainage openings 6 are positioned in the retention means. More specifically, they are positioned in the expansive part of the catheter.

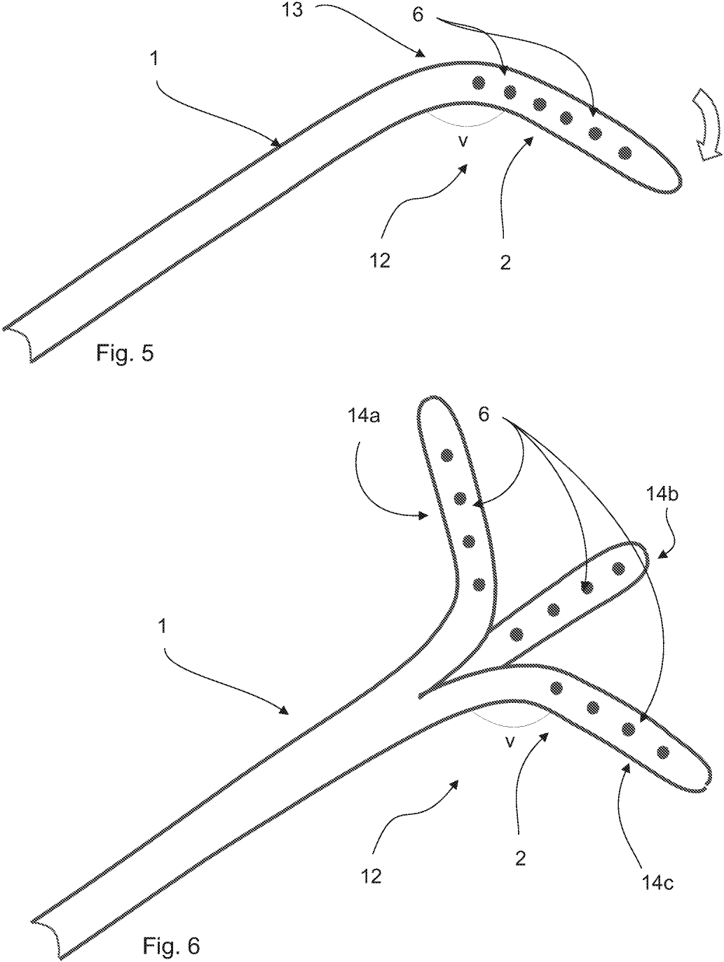

FIG. 5 illustrates an embodiment of a urinary catheter provided with retention means 12 in the form of a bend portion 13 just distally of the tip portion. The bend portion may be made as a particular flexible portion of the catheter and may bend by itself when not subjected to any outside straightening. In the illustrated figures, the tip portion is bend approximately 75 degrees with respect to the tubular portion, such that an angle v between the tubular portion and the tip portion is around 105 degrees.

FIG. 6 illustrates an embodiment of a urinary catheter provided with retention means 12 in the form of three arms 14a, 14b, 14c. The arms constitute the tip portion of the catheter. Furthermore, the drainage openings 6 are positioned in the arms. The arms are at an angle to the tubular portion of the catheter. The angle may be around 75 degrees or less such as 60 degrees.

FIG. 7 illustrates an embodiment of a urinary catheter provided with retention means 12 in the form of a curved proximal portion of the catheter. Such a configuration is sometimes known as a pig-tail. The curved proximal portion may correspond to the tip portion and includes in the illustrated embodiment the drainage portion including the drainage openings 6. The curved proximal portion serves the purpose of retaining the urinary catheter with the tip portion in the bladder for the dwelling period of the catheter.

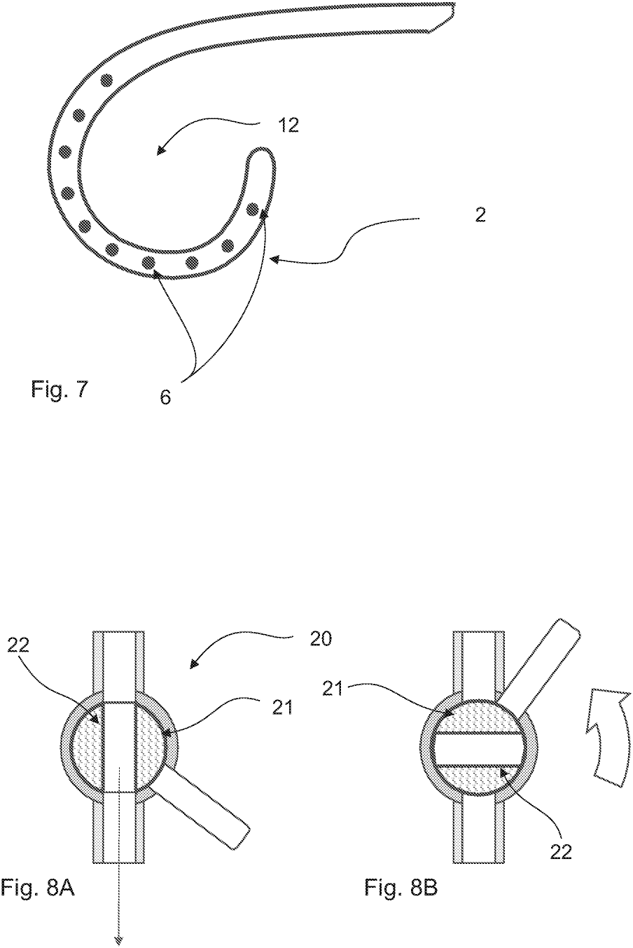

FIGS. 8A and 8B illustrate an embodiment of a valve, that may be used in a closure used to close off the distal end of the interior lumen of a catheter as described herein. The valve 20 is illustrated as a standard ball-valve. The valve 20 has a rotatable ball 21 with a through-going channel 22. The position of the through-going channel can be controlled by a control-lever 23 extending from one side of the ball. The control-lever allows for rotation of the ball such as to position it in an open position with the through-going channel 22 extending in the same direction as the interior lumen and allow for draining of urine through the valve and further to position it in a closed position with the through going channel 22 extending across the direction of the interior lumen, thus closing off from urine draining through the valve. The open position is illustrated in FIG. 8A and the closed position is illustrated in FIG. 8B.

FIG. 9 illustrates the flow-rate through a catheter as a function of the total area of the drainage openings. Curves for three different catheter sizes (CH10, CH12 and CH16) are shown in the figure. The top curve is the curve for a CH16 catheter, the middle curve is the curve for a CH12 catheter, and the lowest curve is the curve for a CH10 catheter. The figure illustrates that the flow-rate converges approximately when the total area of the drainage openings (the total inflow area) is of the same size as the cross-sectional area of the inner lumen of the catheter. This is shown by the vertical lines drawn from where the curves flatten and to the X-axis. The cross-sectional area of the inner lumen of a CH16 is approximately 11 mm², of a CH12 approximately 5.5 mm², and for a CH10 approximately 4 mm².

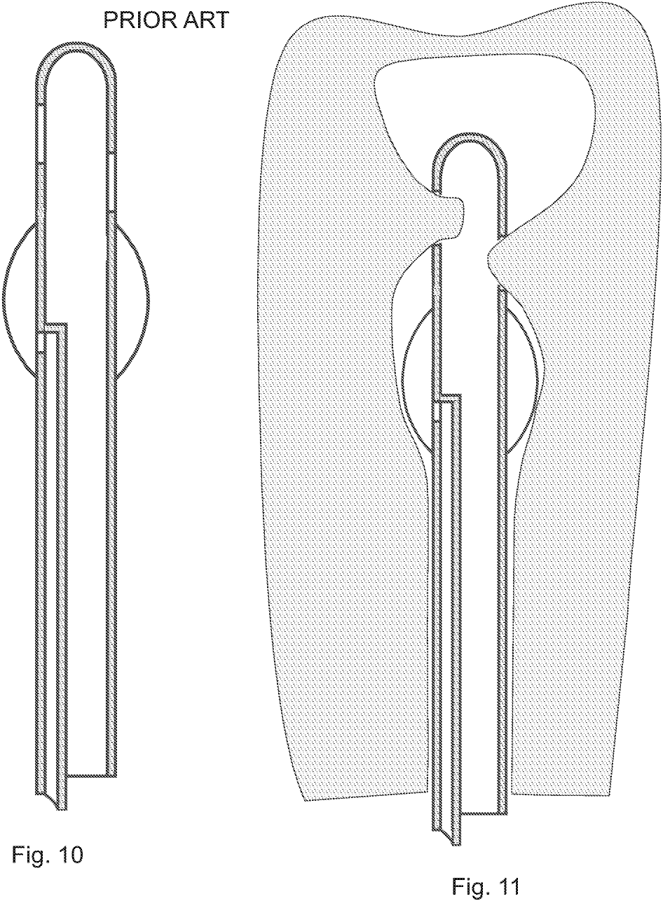

FIGS. 10 and 11 illustrate a prior art catheter, which is not part of this invention. FIG. 10 illustrates a cross-sectional view of a standard Foley catheter, i.e. an indwelling catheter with two large eyelets, which is retained in the bladder by use of an inflatable balloon.

FIG. 11 illustrates a situation that may occur with such a prior art catheter. The figure illustrates that the bladder tissue may enter into one of the eyelets, as the bladder is close to being empty. When one of the standard eyelets is closed off like this, the catheter will continue to provide a suction effect through the other eyelet. Because of the limited room inside a bladder, when it is close to being empty, the other eyelet may come into close contact with bladder tissue. This very flexible and soft bladder tissue may then be sucked into the other eyelet as well — as illustrated in FIG. 11. When this happens, a situation occurs where the bladder tissue will not by itself be able to be removed from the eyelets, because the catheter will still be under sub-pressure. Furthermore, as illustrated in the figure, urine may still be present in the bladder in pockets situated next to or below the blocked eyelets. This urine will then be refrained from being drained out of the bladder.

1-28. (canceled)

29. An indwelling urinary catheter comprising:
a connector;
a tubular portion having a distal end attached to the connector, with the tubular portion extending to and terminating at a tip located at a proximal end of the tubular portion;
a lumen formed in the tubular portion, with the lumen extending through the tubular portion from the tip through the connector;
an inflatable balloon coupled to the tubular portion at a location distal the tip, where the inflatable balloon is adapted to maintain the indwelling urinary catheter in a bladder of a user; and
a proximal portion of the tubular portion extends from inflatable balloon to the tip, where the proximal portion provides a drainage surface of the urinary catheter and includes more than 11 drainage openings formed through a wall of the proximal portion and communicating with the lumen.

30. The indwelling urinary catheter of claim 29, wherein a viscous resistance surface area for the indwelling urinary catheter is T*28 mm, where the viscous resistance surface area is calculated as a product of a thickness T of the wall of the proximal portion multiplied by a sum of circumferences of the more than 11 drainage openings.

31. The indwelling urinary catheter of claim 29, wherein each of the more than 11 drainage openings has a cross-sectional area of less than 2 mm².

32. The indwelling urinary catheter of claim 29, wherein a sum of circumferences of the more than 11 drainage openings is approximately 28 mm.

33. The indwelling urinary catheter of claim 29, wherein a first drain opening of the more than 11 drainage openings has a first area and a second drain opening of the more than 11 drainage openings has a second area, and the first area is different from the second area.

34. The indwelling urinary catheter of claim 29, wherein the drainage surface of the urinary catheter includes between 11 and 100 drainage openings.

35. The indwelling urinary catheter of claim 29, further comprising:
a valve coupled with the lumen and configured to close off a flow of urine through the tubular portion.

36. The indwelling urinary catheter of claim 29, wherein the tip located at the proximal end of the tubular portion is a closed-off tip.

37. The indwelling urinary catheter of claim 29, wherein the tip located at the proximal end of the tubular portion is an open tip.

38. The indwelling urinary catheter of claim 29, wherein the more than 11 drainage openings are distributed along a longitudinal direction of the drainage surface and around a circumference of the drainage surface.

39. The indwelling urinary catheter of claim 29, wherein the more than 11 drainage openings are distributed equidistantly around a circumference of the drainage surface in two longitudinal rows.

40. The indwelling urinary catheter of claim 29, wherein the more than 11 drainage openings are distributed equidistantly around a circumference of the drainage surface in four longitudinal rows.

41. The indwelling urinary catheter of claim 29, wherein the more than 11 drainage openings are distributed equidistantly around a circumference of the drainage surface in 6 longitudinal rows.

42. The indwelling urinary catheter of claim 29, wherein the more than 11 drainage openings are distributed equidistantly around a circumference of the drainage surface in 8 longitudinal rows.

43. The indwelling urinary catheter of claim 29, wherein the more than 11 drainage openings are distributed in a helical distribution around a circumference of the drainage surface.

44. The indwelling urinary catheter of claim 29, wherein the more than 11 drainage openings are distributed equidistantly around a circumference of the drainage surface in longitudinal rows, and a first row of the longitudinal rows is staggered relative to a second row of the longitudinal rows.

45. The indwelling urinary catheter of claim 29, wherein the more than 11 drainage openings are distributed across the drainage surface with more drainage openings formed near the tip than are formed near the inflatable balloon.
